(19) United States
(12) Patent Application Publication
(10) Pub. No.: US 2025/0312358 A1
(43) Pub. Date: Oct. 9, 2025

(54) POWDER TEA COMPOSITION INCLUDING CANNABINOID AND METHOD FOR PRODUCING POWDER TEA COMPOSITION

(71) Applicant: C&H Inc., Kyoto (JP)

(72) Inventor: Hiroki Hirano, Kyoto (JP)

(73) Assignee: C&H Inc., Kyoto (JP)

(21) Appl. No.: 18/999,316

(22) Filed: Dec. 23, 2024

(30) Foreign Application Priority Data
Apr. 5, 2024 (JP) 2024-061253

(51) Int. Cl. A61K 31/00; A23F 3/14; A61K 9/00; A61K 36/185; A61K 47/36; A61K 47/46

(52) U.S. Cl. CPC A61K 31/658; A23F 3/14; A61K 9/0095; A61K 36/3482; A61K 47/36; A61K 47/46

(57) ABSTRACT

Provided is a powder tea composition which enables a cannabinoid-containing food and beverages having a high tea leaf flavor and being capable of sufficiently enjoying the effect of cannabinoid to be produced. Provided is a powder tea composition in a powder form, including a cannabinoid-containing water-soluble granulated product and a tea leaf-ground product.

### TECHNICAL FIELD

The present invention relates to a powder tea composition including cannabinoid and a method for producing a powder tea composition. More specifically, the present invention relates to, for example, a powder tea composition to be used in a food and beverages such as a beverage to be dissolved or extracted in an aqueous medium such as water or hot water and then drunk.

### BACKGROUND ART

Cannabinoid belongs to a group of lipophilic, physiologically active substances mainly extracted from hemp plants, and is known to interact with receptors in the brain or body to exert a neurotropic effect and/or an analgetic action. In particular, cannabidiol (CBD) is cannabinoid attracting considerable attention because it is known to exhibit an anti-inflammatory effect, an antioxidant effect, an anticonvulsive action, a neuroprotective action, an anti-anxiety action and the like and it is high in tolerability and is not confirmed with respect to dependence. Cannabinoid has these effects, and therefore may be used in beverages and/or foods.

As a beverage using cannabidiol, there is known CBD tea including CBD oil having lipid solubility and a tea leaf (see Patent Literatures 1 to 3). There are also known commercially available products of a tea beverage including CBD and powder tea including CBD.

### CITATION LIST

### Patent Literature

[Patent Literature 1] Japanese Translation of PCT International Application Publication No. 2019-501626
[Patent Literature 2] Japanese Translation of PCT International Application Publication No. 2019-505163
[Patent Literature 3] Japanese Translation of PCT International Application Publication No. 2021-526126

### SUMMARY OF INVENTION

### Technical Problem

Cannabinoid tea including cannabinoid and a tea leaf is used as a tea beverage in which a tea leaf is dipped in hot water to extract a tea leaf-derived component. In a case where the tea beverage is in a state of a liquid including cannabinoid, cannabinoid can be decomposed or degraded during storage to cause a decrease in the content of cannabinoid, volatilization or degradation of a tea flavor, and loss in taste, and therefore the property of allowing for storage in a solid powder form is preferred.

Conventionally, cannabinoid does not uniformly disperse due to lipid solubility thereof even if powder tea in which cannabinoid is directly used is dissolved in hot water. There has been then carried out a method involving mixing cannabinoid and a tea powder together with an emulsifier or the like. However, a tea powder of cannabinoid obtained by such a mixing method using an emulsifier has been produced by mixing a tea leaf and cannabinoid by a solvent such as oil or an emulsifier and thus a problem has been that a drying step is needed so that a tea taste or the like may degrade. In addition, if cannabinoid and a tea powder are mixed in a dry state with a solid additive having the emulsification action, without any solvent, low fluidity of cannabinoid and such a tea powder has made suppression of clump formation and rapid uniform mixing difficult, and/or cannabinoid and an emulsifier have not been uniformly mixed by a solvent, thereby making sufficient dispersion of cannabinoid in water difficult. As a result, it has been difficult to keep uniformity of the grain size of a tea powder in the form of a food and beverage product and achieve stable dissolution of cannabinoid. From the foregoing, a conventional tea beverage including cannabinoid has not been able to sufficiently enjoy the effects of a tea leaf flavor and cannabinoid.

Accordingly, an object of the present invention is to provide a powder tea composition which can allow for production of a cannabinoid-containing food and beverages having a high tea leaf flavor and being capable of sufficiently enjoying the effect of cannabinoid.

### Solution to Problem

The present inventor has made intensive studies in order to achieve the above object, and as a result, has found that a cannabinoid-containing food and beverages having a high tea leaf flavor and being capable of sufficiently enjoying the effect of cannabinoid can be produced by a specified powder tea composition. The present invention has been completed based on such findings.

In other words, the present invention provides a powder tea composition including a cannabinoid-containing water-soluble granulated product or a raw material capable of forming the water-soluble granulated product, and a tea leaf powder.

The raw material capable of forming the water-soluble granulated product preferably includes water-soluble cannabinoid and a granulated powder.

A content rate of the water-soluble granulated product and/or the raw material capable of forming the water-soluble granulated product is preferably 1% by mass or more based on a total amount of 100% by mass of the powder tea composition.

The tea leaf powder preferably includes a tea leaf powder of a plant in the genus Camellia of the family Theaceae.

The present invention provides a method for producing a powder tea composition, the method including mixing/stirring a composition including a water-soluble granulated product of a lipid-soluble compound and/or a raw material capable of forming the water-soluble granulated product, and a tea leaf powder, by a container movement system.

### Advantageous Effect of Invention

According to the powder tea composition of the present invention, a cannabinoid-containing food and beverages having a high tea leaf flavor and being capable of sufficiently enjoying the effect of cannabinoid can be produced.

### DESCRIPTION OF EMBODIMENTS

### [Powder Tea Composition]

The powder tea composition of the present invention includes at least a water-soluble granulated product of cannabinoid, or a raw material capable of forming the water-soluble granulated product, and a tea leaf powder. In other words, the powder tea composition of the present invention may include a water-soluble granulated product of cannabinoid and a tea leaf powder, may include a raw material capable of forming the water-soluble granulated product of cannabinoid, and a tea leaf powder, or may be include a water-soluble granulated product of cannabinoid, a raw material capable of forming the water-soluble granulated product, and a tea leaf powder. In the powder tea composition of the present invention, cannabinoid is uniformly dispersed in the powder tea composition. A tea leaf powder is used in the powder tea composition of the present invention, and thus, when a tea beverage in which the powder tea composition is dissolved in an aqueous solvent such as hot water is taken, a tea leaf flavor can be high and a taste originating from tea can be felt. The powder tea composition is provided and thus formed into a tea beverage immediately before intake, and therefore degradation of a tea leaf flavor and cannabinoid can be kept to the minimum.

Cannabinoid is lipid-soluble and then the water-soluble granulated product has fluidity and water solubility increased, for example, by addition of additive(s) such as a binder and/or an excipient having a granulation action, and therefore cannabinoid itself is not used singly, but used as the water-soluble granulated product, or water-soluble cannabinoid is used as a raw material capable of forming the water-soluble granulated product, thereby enabling clump (aggregate) formation to be kept to the minimum and also enabling a uniform dispersion with a grain size more stabilized, with cannabinoid as a core, to be obtained, during powder mixing of cannabinoid and a tea leaf powder is carried out, and for example, allowing cannabinoid to be rapidly uniformly dispersed in a solution in a tea beverage in which the powder tea composition is dissolved in an aqueous solvent. In addition, neither a liquid form, nor a slurry form is made during mixing with a tea leaf, and thus a drying step is not needed and a tea taste is not impaired. Furthermore, cannabinoid is processed so as to be water-soluble and thus the absorption efficiency of cannabinoid into the body is also excellent. In addition, not a simple powder, but a granulated product (for example, granule) can be provided to result in a reduction in taste-masking effect by flouriness. Thus, the powder tea composition of the present invention can be used in a food and beverages, to allow a tea leaf flavor to be kept to the maximum, and also allow the effect of cannabinoid to be sufficiently enjoyed and allow cannabinoid to be efficiently taken in the body. Furthermore, the water-soluble granulated product can be used to hardly cause clump formation by static electricity and suppress aggregation of the powder tea composition, thereby allowing a powder tea composition dispersed at a uniform grain size to be rapidly obtained and also be excellent in handleability.

### (Tea Leaf Powder)

Herein, the “tea leaf powder” means a tea leaf ground (tea leaf-ground product), and is distinguished from a component “tea leaf extract” extracted from such a tea leaf. The tea leaf used here can be a leaf obtained from a plant (Camellia sinensis (L) O. Kuntze, or the like) in the genus Camellia of the family Theaceae, or its subspecific, varietal, crossbred or clone species, or a leaf of a plant to be formed into a Rooibos or herb tea capable of being drunk as a decoction. The tea leaf powder is obtained by subjecting a new bud, a raw leaf, or the like to plucking and thereafter crude tea processing/tea production processing, and grinding the resultant into a powder form. Such grinding can be performed by a known or traditional method with a stone mill, a ball mill, a jet mill, a pin mill, an airflow-type grinder, or the like. The tea leaf powder may be used singly or in combinations of two or more kinds thereof. While a leaf is exemplified as a representative of a raw material of the tea leaf powder, any other moiety such as a bud or a stem can also be appropriately used. The tea leaf powder preferably includes a tea leaf-derived tea powder raw material cultivated in Japan.

The tea leaf powder preferably includes one obtained by only a physical treatment and/or a biological treatment such as steaming (sweating, boiling), wilting, toasting (pan-toasting), kneading (coarse kneading, rolling, middle kneading, precise kneading), sieving, loosing, fermentation (oxidation, addition reaction, microbial fermentation), cooling, drying, blowing (warm air), dehydration, drying (sun-drying), light-shielding, humidifying, heat-retention (warming), roasting (dry roasting), leaf beating, cutting, pulverizing, grinding, stirring, mixing, forming, form-finishing, and combining (tea leaf blending), in steps until powdering after plucking, such as crude tea processing, tea production processing, and grinding processing.

The tea leaf (plant in the genus Camellia of the family Theaceae) can be classified into non-fermented tea, semi-fermented tea, fermented tea, and post-fermented tea depending on the processing method. Here, fermentation with respect to the above non-fermented tea, semi-fermented tea, and fermented tea critically indicates not a treatment with microorganisms, but oxidation by an enzyme in the tea leaf. On the other hand, fermentation with respect to the post-fermented tea typified by black tea indicates fermentation in a scientifically correct sense because there is an aging step with microorganisms. Examples of the non-fermented tea include green tea such as crude tea, Sencha, Gyokuro, covered tea, dried tea (powdered green tea), coarse green tea, roasted green tea, tea in pan, stem tea, stick tea, and bud tea. Examples of the semi-fermented tea include oolong tea such as Tekkannon, Shikisyu, Ogonkei, and Buisuisen tea. Examples of the fermented tea include red tea such as Darjiling, Assam, Sri Lanka, and Japanese red tea. The tea leaf is preferably a tea leaf domestically produced. Herein, the “domestically produced” refers to one cultivated in Japan.

The tea leaf powder preferably includes a tea leaf powder of a plant in the genus Camellia of the family Theaceae. Tea derived from the genus Camellia is supposed to be able to be suitable for improvement of lifestyle-related diseases, and is seen to have a synergistic effect with cannabinoid. For example, green tea is rich in theanine and is seen to have a relax effect, and also includes nutrients such as vitamins. In particular, roasted green tea is rich in pyrazine instead of theanine and is small in amount of caffeine, and therefore can be expected to have a high relax effect by combination with CBD and is more excellent in sleep improvement effect.

The average grain size of the tea leaf powder is not particularly limited, and is, for example, 1 to 180 μm, preferably 2 to 150 μm, more preferably 3 to 100 μm, or may be 5 to 80 μm, or 10 to 50 μm. The average grain size can be appropriately adjusted by appropriately selecting the above grinding unit, and/or performing classification with a sieve or a classifier.

### (Water-Soluble Granulated Product)

The water-soluble granulated product is a granulation-processed powder including at least cannabinoid. The cannabinoid is uniformly dispersed in the granulated product. The granulation-processed powder means a powder improved in grain size of the powder before and after granulation, and therefore the average grain size is not particularly limited. Here, a water-soluble (water-dispersible) cannabinoid-containing powder obtained by adding a component capable of serving as a binder described below and uniformly powdering the resultant corresponds to the water-soluble granulated product. Such uniform dispersing can be confirmed by HPLC or the like, the concentrations of cannabinoid detected in respective solutions which are prepared with a plurality of samples collected from any points in the powder so as to be at the same concentrations by solvents are comparable, and the difference therebetween is ±10% or less, preferably ±5% or less, more preferably ±3% or less. The water-soluble granulated product may be used singly or in combinations of two or more kinds thereof.

The water-soluble granulated product is not confirmed to be phase-separated in mixing at a soluble or dispersible concentration with water. Therefore, uniform dispersibility in an aqueous solvent is excellent. Such phase separation is preferably not confirmed even after a lapse of 2 hours or more, 3 hours or more, half a day or more, one day or more, or two days or more at room temperature.

The repose angle of the water-soluble granulated product is preferably 45° or less, more preferably 40° or less, further preferably 35° or less. In a case where the repose angle is 45° or less, mixing in the tea leaf powder is more facilitated and clump (aggregate) formation in the powder tea composition of the present invention can be more suppressed.

Herein, the repose angle can be measured by a known injection method. For example, a cone-shaped deposited product is formed by dropping the water-soluble granulated product through a funnel onto a horizontal surface and the value of elevation angle of the deposited product can be determined as the repose angle.

Example of the cannabinoid include cannabidiol (CBD), cannabidivarin (CBDV), cannabigerol (CBG), cannabinol (CBN), tetrahydrocannabinol (THC), hydroxyhexahydrocannabinol (HHC), tetrahydrocannabivarin (THCV), cannabichromene (CBC), and cannabidiolic acid (CBDa). In particular, CBD is preferred. The water-soluble granulated product may include only one cannabinoid, or may include two or more kinds of cannabinoid.

The content rate of cannabinoid in the water-soluble granulated product is preferably 0.1% by mass or more, more preferably 0.5% by mass or more, or may be 1% by mass or more based on a total amount of 100% by mass of the water-soluble powder from the viewpoint that the effect of cannabinoid is more sufficiently enjoyed. The content rate is not particularly limited, and is preferably 60% by mass or less.

The recovery rate of the water-soluble granulated product, as quantitatively determined by HPLC analysis with respect to a solution prepared by dissolution in a solvent, is preferably 90% or more, more preferably 95% or more, further preferably 97% or more. The solution, which is prepared by appropriately carrying out, if necessary, a pre-treatment for removal of a precipitate by concentration adjustment, filtration and/or the like performed before HPLC, can be used. The recovery rate is calculated by the following expression. It is determined that, as the recovery rate is higher, the amount of cannabinoid in the powder tea composition is closer to the amount of cannabinoid compounded. It is also determined that, if the accuracy of the recovery rate is low, the accuracy of the value calculated of the degree of dispersion of cannabinoid in the powder tea composition, described below, is low.

$$Recovery rate [ \% ] = [ Amount of cannabinoid detected ( parts by mass ) / Amount of cannabinoid loaded ( parts by mass ) \times 100 ]$$

The water-soluble granulated product is produced by granulation processing, and is a grain granulated according to a known or traditional granulation method. The granulation method is not particularly limited, and examples include dry granulation (slugging (flat plate compaction), roller compaction, pneumatic dry granulation, melt granulation, and the like), wet granulation (high shear granulation, fluid bed granulation, low shear granulation, vapor granulation, foaming granulation, twin (biaxial) screw granulation, and the like), freezing granulation (freeze-drying), spray-drying, spray coagulation/freezing, extrusion and subsequent spheroidizing. There is also included a method including adding a component capable of serving as a binder described below and uniformly powdering the resultant. The granulation method may be adopted singly or in combinations of two or more kinds thereof.

The water-soluble granulated product may include a granulation additive (binder) in order to enhance fluidity and solubility in water. The water-soluble granulated product may also be one that does not include any granulation additive (binder) and that is obtained by adding at least one selected from the group consisting of an emulsifier, a surfactant, a solubilizer, a dispersant, a stabilizer, a thickener, a coagulant, a gelling agent, an excipient, and any other hydrophilic polymer, and then performing granulation processing.

The binder is not particularly limited, and examples include polysaccharide, a protein, a vinyl polymer, polyether, oligosaccharide, sugar, fatty acid ester, wax, one classified into any other dietary fiber, and a hydrophilic polymer. Examples of the polysaccharide include gum acacia, gum arabic, agar, tragacanth gum, sodium alginate (alginate), carrageenan, xanthan gum, guar gum, dextran, pullulan, pectin, chitosan, inulin, hemilose, a cellulose-based polymer, and starch. Examples of the cellulose-based polymer include carmellose or any salt thereof, hydroxypropylcellulose, hydroxypropylmethylcellulose, methylcellulose, hydroxyethylcellulose, ethylcellulose, and microcrystalline cellulose. Examples of the starch include starch (wheat starch, corn starch, potato starch, and the like), pregelatinized starch, dextrin, cyclodextrin, carboxymethyl starch, hydroxypropyl starch, hydroxyethyl starch, and partially pregelatinized starch. Examples of the protein include animal proteins such as gelatin and purified gelatin, and vegetable proteins such as a soybean protein. Examples of the vinyl polymer include polyvinylpyrrolidone, polyvinyl alcohol, copovidone, and an acrylic polymer. Examples of the acrylic polymer include polyacrylic acid, a carboxyvinyl polymer, sodium polyacrylate, and polymethyl methacrylate. Examples of the polyether include polyethylene glycol (macrogol). Examples of the sugar include white sugar starch syrup, fructose, sugar alcohols (for example, xylitol, sorbitol, maltitol, and mannitol), glucose, lactose, and sucrose. Examples of the fatty acid ester include mono- or diglyceride of fatty acid. Examples of the wax include beeswax. The binder may be used singly or in combinations of two or more kinds thereof.

The water-soluble granulated product may include, in addition to the binder, any additive such as an emulsifier, a surfactant, a solubilizer, a dispersant, stabilizer, a thickener, a coagulant, a gelling agent, an excipient, an oxidation inhibitor, an antioxidant, any other hydrophilic polymer, and/or a tea leaf (tea leaf powder and/or the like), in order to further enhance solubility in water and other functionality. The additive may be used singly or in combinations of two or more kinds thereof.

As the oxidation inhibitor, for example, known or traditional one to be added as an oxidation inhibitor to a food product can be used, and examples include vitamin C and tocopherol.

As the antioxidant, known or traditional one to be added as an antioxidant to a food product and then used can be used, and examples include various vitamins such as vitamins E, A, and C, beauty components such as astaxanthin, lutein, ceramide and collagen, and oils or extracts including these.

The water-soluble granulated product may include any additive such as a food additive that can be included in food and beverages and/or pharmaceutical products. Examples of the additive include an expansion agent such as a baking powder, a colorant, a color former, a bleacher, a sweetener, a bittering agent, a sour agent, a perfume, a seasoning agent, an antiseptic agent, a bactericidal agent, a mildew-proofing agent, a preservation agent, a nutrient enhancer, and an enzyme. The additive may be used singly or in combinations of two or more kinds thereof. The powder tea composition of the present invention and a food and beverages described below may include any additive exemplified and described as the additive that can be included in the water-soluble granulated product.

As described above, the powder tea composition of the present invention may include a raw material capable of forming the water-soluble granulated product instead of the water-soluble granulated product or together with the water-soluble granulated product. The raw material capable of forming the water-soluble granulated product includes, for example, water-soluble cannabinoid and a granulated powder. Examples of the powder tea composition of the present invention, which includes water-soluble cannabinoid and a granulated powder, one obtained by adding the tea leaf powder to a powder A made by mixing water-soluble cannabinoid (powder including cannabinoid, which does not improve the repose angle as compared with single cannabinoid, which also does not include any binder, and which is not produced through a common granulation process) with the granulated product, and mixing the resultant. Herein, the powder A is observed to be improved in repose angle and/or enhanced in grain size as compared with the original water-soluble cannabinoid, due to the influence of mixing with the granulated product.

The water-soluble cannabinoid is not confirmed to be phase-separated in mixing at a soluble or dispersible concentration with water. Such phase separation is preferably not confirmed even after a lapse of 2 hours or more, 3 hours or more, half a day or more, one day or more, or two days or more at room temperature. The water-soluble cannabinoid may be included singly or in combinations of two or more kinds thereof.

The granulated powder is a granulation-processed powder, and is a grain granulated according to a known or traditional granulation method. Examples of the granulation method include any method exemplified and described as the granulation method for obtaining the above water-soluble granulated product. The granulation method may be adopted singly or in combinations of two or more kinds thereof.

The granulated powder may include a granulation additive (binder) in order to enhance fluidity and solubility in water. The granulated powder may also be one that does not include any granulation additive (binder) and that is obtained by adding at least one selected from the group consisting of an emulsifier, a surfactant, a solubilizer, a dispersant, a stabilizer, a thickener, a coagulant, a gelling agent, an excipient, and any other hydrophilic polymer, and then performing granulation processing. Examples of the binder include any binder exemplified and described as the binder usable in granulation processing of the above water-soluble granulated product.

Specific examples of the granulated powder include the water-soluble granulated product, from which cannabinoid is removed, and a tea powder (for example, powder in which a binder and/or an emulsifier are/is added to an extracted liquid of tea and granulation is performed) granulated with a tea leaf (tea leaf powder or the like) as a raw material.

### (Powder Tea Composition)

The powder tea composition may include any other component than the water-soluble granulated product, the raw material capable of forming the water-soluble granulated product, and the tea leaf powder. Examples of such other component include any component exemplified as any other component that can be included in the above water-soluble granulated product, any component exemplified above as the granulated powder, a preservation agent, a pH adjuster, a sweetening agent, a nutrient enhancer, a thickening stabilizer, a dietary fiber, a quality stabilizer, and acid. Such other component may be used singly or in combinations of two or more kinds thereof.

The powder tea composition is in a powder form. The powder tea composition may be a collected product of primary grains, or may be a collected product of aggregates of a plurality of grains.

The powder tea composition preferably includes a core-shell powder with the water-soluble granulated product as a core layer and the tea leaf powder as a shell layer. In a case where such a core-shell structure is included, the powder tea composition can be much more inhibited from being excessively aggregated (clump formation).

The content rate of the tea leaf powder in the powder tea composition is preferably 20% by mass or more, more preferably 30% by mass or more, further preferably 40% by mass or more based on a total amount of 100% by mass of the powder tea composition. In a case where the content rate is 20% by mass or more, a tea leaf taste can be sufficiently felt. The content rate is, for example, 99% by mass or less, preferably 95% by mass or less, more preferably 90% by mass or less, further preferably 80% by mass or less. In a case where the content rate is 99% by mass or less, clump formation tends to be more suppressed, and ease of handling of the powder tea composition and uniform dispersibility of cannabinoid are more excellent.

The content rate of the water-soluble granulated product and/or the raw material capable of forming the water-soluble granulated product in the powder tea composition is preferably 1% by mass or more, more preferably 5% by mass, more preferably 10% by mass or more, further preferably 20% by mass or more based on a total amount of 100% by mass of the powder tea composition. In a case where the content rate is 1% by mass or more, ease of handling of the powder tea composition and uniform dispersibility of cannabinoid are more excellent. The content rate is, for example, 80% by mass or less, preferably 70% by mass or less, more preferably 60% by mass or less. In a case where the content rate is 80% by mass or less, a tea leaf taste can be sufficiently felt.

The content rate of cannabinoid in the powder tea composition is preferably 0.05% by mass or more, more preferably 0.1% by mass or more, further preferably 0.3% by mass or more based on a total amount of 100% by mass of the powder tea composition. In a case where the content rate is 0.05% by mass or more, the effect of cannabinoid can be sufficiently expected. The content rate is, for example, 30% by mass or less.

The degree of dispersion of cannabinoid in the powder tea composition of the present invention is preferably 90% or more, more preferably 95% or more, further preferably 97% or more. The degree of dispersion can be determined by dissolving the powder tea composition in a solvent, subjecting the prepared solution to quantitative determination with HPLC analysis, and performing calculation according to the following expression. The solution, which is prepared by appropriately carrying out, if necessary, a pre-treatment for removal of a precipitate by concentration adjustment, filtration and/or the like performed before HPLC, can be used, and specifically, the same pre-treatment as the pre-treatment with respect to the recovery rate is carried out. The degree of dispersion is an index for determining whether or not cannabinoid is uniformly dispersed in the resulting powder tea composition, and it is indicated that, as the degree of dispersion is closer to 100%, cannabinoid is uniformly dispersed.

$$Degree of dispersion ( \% ) = 100 \times ( Cs - \vert Cs - C \vert ) / Cs$$

C: Amount of cannabinoid detected by HPLC analysis of powder tea composition arbitrarily collected in amount of 1/10 or less (preferably 1/100 or less) of amount of production
Cs: Amount of cannabinoid detected by HPLC analysis of reference standard sample with raw materials mixed for providing the same compositional ratio as that of powder tea composition

The powder tea composition of the present invention can be produced by a known or traditional method. For example, raw materials of the powder tea composition are loaded in a container, and mixed/stirred, to produce the composition. A known or traditional method can be adopted for such mixing/stirring, and examples include mixing/stirring by a high shear mixer, a high shear mixer, a homomixer, a ball mill, a bead mill, a container movement system, a high-pressure homogenizer, a ultrasonic homogenizer, a colloid mill, or a stirrer. A container movement system is preferably adopted for such mixing/stirring from the viewpoint that degradation in taste due to heat is avoided and the powder tea composition excellent in uniform dispersibility of the water-soluble granulated product is easily obtained. The container movement system is a system for mixing/stirring by vibration/shaking of a container, rotation of a container, or the like. The order of loading to the container is not particularly limited, and the water-soluble granulated product is preferably loaded before loading of the tea leaf powder. Such an order of loading can be adopted to produce the powder tea composition including the core-shell powder with the tea leaf powder attached to a surface of the water-soluble granulated product. In addition, clump formation can be more suppressed and uniform dispersibility of the water-soluble granulated product is excellent. In the production method, no solvent is preferably added from the viewpoint that, when the powder tea composition is obtained, a drying step is not underwent and thermal degradation is prevented and from the viewpoint that volatilization is prevented from being promoted.

Any other lipid-soluble compound than cannabinoid can be used instead of cannabinoid to produce a powder tea composition in the same manner as in the powder tea composition including cannabinoid. In other words, the present invention provides a method for producing a powder tea composition, the method including mixing/stirring a composition including a water-soluble granulated product of a lipid-soluble compound and/or a raw material capable of forming the water-soluble granulated product, and a tea leaf powder, by a container movement system. The water-soluble granulated product and the raw material capable of forming the water-soluble granulated product in this paragraph, used here, can be those in which “cannabinoid” is replaced with “lipid-soluble compound”, in the above cannabinoid-containing water-soluble granulated product and the above raw material capable of forming the water-soluble granulated product, exemplified and described. Even in the case of a lipid-soluble compound other than cannabinoid, the lipid-soluble compound can be used as a water-soluble granulated product or the lipid-soluble compound subjected to water-soluble processing can be used with the granulated powder, to obtain the effect in which the lipid-soluble compound can be uniformly powdery mixed with the tea leaf powder even if not formed into a liquid or slurry and furthermore the lipid-soluble compound is uniformly dispersed in a tea beverage of the powder tea composition dissolved in an aqueous solvent. Since neither a liquid, nor a slurry is formed, the effect in which a drying step is not needed and a tea taste is not impaired is also obtained. Thus, there is obtained a powder dispersion with a stable grain size, in which a tea taste is not impaired and the lipid-soluble compound serves as a core. The “water-soluble granulated product” and “water-soluble processed lipid-soluble compound” are those that are not observed to be phase-separated in mixing in a soluble or dispersible amount with water, and such phase separation is preferably not confirmed even after a lapse of 2 hours or more, 3 hours or more, half a day or more, one day or more, or two days or more at room temperature.

The powder tea composition of the present invention can be contained in a food and beverages (beverage and food product). In other words, the present invention can provide a food and beverages containing the powder tea composition of the present invention. The powder tea composition of the present invention is preferably contained in an aqueous solvent (water, hot water, or the like) and thus formed into a beverage, and is most preferably dissolved in water, hot water or the like and then drunk as a tea beverage. In this regard, the powder tea composition of the present invention can also be provided in the form of fast-food powder tea (instant powder tea). The tea beverage includes non-fermented tea, semi-fermented tea, and fermented tea, and specific examples include tea, for example, non-fermented tea (green tea) produced by steaming, such as Sencha, coarse green tea, roasted green tea, Gyokuro, covered tea, and sweet tea; non-fermented tea, for example, pan-fired tea such as Ureshinocha, Aoyagicha, and each Chinese tea; semi-fermented tea such as paochung tea, Tekkannon tea, and oolong tea; fermented tea such as red tea; and post-fermented tea such as Awa coarse green tea, pu-erh tea.

In a case where the powder tea composition of the present invention is contained in an aqueous solvent, the content rate of the powder tea composition of the present invention in the solution is not particularly limited, and is, for example, 0.05 to 30% (w/v), preferably 0.1 to 25% (w/v), more preferably 0.2 to 20% (w/v), further preferably 0.25 to 15% (w/v).

In a case where the powder tea composition of the present invention is contained in an aqueous solvent, the content rate of cannabinoid in the solution is not particularly limited, and is preferably 1 to 500 mg.

The powder tea composition of the present invention may be extracted in an aqueous medium such as water or hot water and then drunk. In this regard, the powder tea composition of the present invention can also be provided as a tea bag in which the powder tea composition is encapsulated. If an insoluble component that can be included in the powder tea composition is not leaked out of a tea bag in dipping of the tea bag in an aqueous medium, only an extracted component can be taken. While it takes time to extract caffeine and catechin from the tea leaf powder even in hot water at high temperature, the water-soluble granulated product of cannabinoid is uniformly included in the powder tea composition of the present invention and therefore cannabinoid can be efficiently taken even in the case of extraction. The temperature of the tea beverage can be lower to extract more cannabinoid and on the other hand decrease the amount of extraction of a tea leaf-derived component such as caffeine, and, for example, the effect on sleeping can be expected to be increased.

The powder tea composition of the present invention can also be added to a food product. The food product may be any of Japanese confectionery and Western confectionery, can be used for confectionery such as cake, sponge cake, candies, cookies, jelly, pudding, or chocolate, frozen dessert such as ice cream, iced lollies, or sherbert, or munchies, and can also be used for bread, a dairy product, protein, or the like. The powder tea composition of the present invention may be mixed with the food product, or may be added to the food product immediately before intake or during intake.

The powder tea composition of the present invention is preferably packed in a container. The container in which the powder tea composition of the present invention is accommodated may have any shape, and examples include a tea canister-shaped container and a stick-shaped container, and a pouch. A container for refilling can also be used. The container preferably has light-shielding and sealing ability from the viewpoint that storage stability is excellent.

The powder tea composition of the present invention can allow a food and beverages having a tea leaf flavor, having a stable content of cannabinoid, and having excellent uniform dispersibility to be produced. Therefore, the powder tea composition of the present invention can try to achieve both a flavor component of terpene derived from the tea leaf powder and the effect of cannabinoid, and achieve the synergistic effect (Entourage effect). The powder tea composition of the present invention is excellent in, for example, cannabinoid dispersibility in an aqueous solvent, storage stability, efficacy (efficacy of tea component and/or cannabinoid), cannabinoid absorption efficiency, and the like. In particular, the water-soluble component can be expected to be enhanced in bioavailability (cannabinoid absorption rate) four times as compared with intake of conventional cannabinoid oil. A product in a form capable of being stored in a powder form can be provided to impart storage stability, for example, even after storage at room temperature (15 to 25° C.) for 300 days, or even in a severe test (after one week at 60° C.), and the content of cannabinoid is not reduced or almost not reduced. In addition, storage stability is excellent to allow the maximum cannabinoid efficacy, absorption rate, and concentration to be stably retained, and also allow a tea taste to be confined until just before intake, and therefore the flavor and the nutrient content of a tea beverage, namely, the taste and the efficacy thereof can be drawn out to the maximum.

Examples of the effect of cannabinoid include an antibacterial effect, an antioxidant effect (oxidation inhibition function), and an anti-inflammatory effect. Specified cannabinoid (for example, CBD, CBN) has been reported to exert a sleep improvement effect. For example, a tea leaf powder of the genus Camellia include not only water-soluble nutrients (caffeine, catechin (polyphenol), theanine (amino acid), GABA, vitamin C, vitamin B, other minerals, polysaccharide, and the like), but also water-insoluble or poorly water-soluble nutrients (β-carotene, vitamin E, co-enzyme Q10, minerals, lipid-soluble dietary fiber, and the like) included in the tea leaf powder. Therefore, the powder tea composition including cannabinoid and the tea leaf powder can also achieve the effect by combination of cannabinoid and the tea leaf powder. In the powder tea composition of the present invention, cannabinoid can prevent oxidation of chlorophyl and catechin included in a powder tea leaf, and an enhancement in preservability can be expected. According to the effect of storage stabilization of cannabinoid, discoloration of green tea, flavor degradation of red tea, and darkening due to catechin polymerization can be expected to be prevented. For example, CBD exerts an excellent antioxidant effect. Vitamin C, while has an antioxidant effect and thus may be additionally added for an enhancement in preservability and/or an enhancement in antioxidant effect in the body, has the problem of increasing a sour taste, whereas CBD has a higher antioxidant effect and has a slighter bitter taste and therefore is more useful as an anti-oxidation component in powder tea. Most of tea originally contains a bitter component, and a slight bitter taste of CBD is compatible with tea and easily tuned therewith.

When the powder tea composition of the present invention is dissolved and stirred in an aqueous solvent such as hot water, cannabinoid is dispersed within at least one minute or several minutes, preferably cannabinoid is dispersed within several seconds to several tens of seconds and thus dispersibility is excellent, and cannabinoid dissolved is excellent in stability preferably for several hours to one day or more. If dispersibility in an aqueous solvent is high, the cannabinoid absorption efficiency is also excellent.

### EXAMPLES

Hereinafter, the present invention is described in more detail with reference to Examples, but the present invention is not limited to these Examples at all. Unless particularly noted, “%” means “% by mass”.

### Example 1

A cylindrical plastic container was charged with about 40% of a commercially available water-soluble CBD powder (granulated product granulated mainly with polysaccharide as a binder, repose angle about: 30°, CBD: 2%), and then charged with about 60% of a domestically produced, blended roasted green tea powder, and these were mixed by vibration and rotation of the container at room temperature for about 60 minutes, to obtain a powder tea composition. In this mixing, clump formation was not confirmed.

### <Degree of Dispersion>

Weighed was 0.1 g of the powder tea composition obtained in Example 1, the composition was loaded to water at about 23° C. so that the CBD concentration was 40 ppm, and stirred, thereafter acetonitrile was added thereto so that CBD was 20 ppm, and stirred, a tea leaf-derived insoluble fraction was removed and then filtration with a membrane filter was performed, and the resulting filtrate was subjected to quantitative determination by HPLC analysis. The degree of dispersion was calculated according to the following expression. As a result, the degree of dispersion was more than 99%, and CBD was evaluated to be uniformly dispersed in the powder tea composition. No clump was formed and the tea leaf was uniformly dispersed in the water-soluble granulated product, and a powder having a uniform grain size was formed in the present powder tea composition.

$$Degree of dispersion ( \% ) = 100 \times ( Cs - \vert Cs - C \vert ) / Cs$$

### <Taste Sense/Feeling Evaluation>

A tea beverage was produced by dissolving 1 g (containing 8 mg of CBD) of the powder tea composition obtained in Example 1 in 200 mL of hot water, and taste-tested before sleeping. As a result, a tea leaf flavor was high and a taste was favorable, and a sense of relaxation and sleepiness were felt, thereby making it possible to have deep sleep. It was also reported from questionary investigation (n=129) carried out that sleep was improved in seventy percent or more of users.

### <Storage Stability>

The powder tea composition obtained in Example 1 was stored at room temperature (15 to 25° C.) for 300 days and thereafter the content of CBD was confirmed after a severe test (at 60° C. for one week), and thus it was determined that the amount of reduction was almost within a measurement error and the reduction was not confirmed or the reduction was almost not observed and was within an allowable range (<5%).

The powder tea composition obtained in Example 1 did not undergo a drying step in production thereof, and was evaluated to have a high tea leaf flavor and a favorable taste as shown in the taste sense/feeling evaluation. The amount of reduction in content of CBD before and after storage was within an allowable range as shown in the above evaluation of storage stability, and it was evaluated that the effect of CBD could be sufficiently enjoyed. The powder tea composition obtained in Example 1 did not cause the clump formation during mixing, and it was evaluated that CBD was uniformly dispersed in the powder tea composition, and that a powder having a uniform grain size was obtained. It can be seen from these that a cannabinoid-containing food and beverages having a high tea leaf flavor and being capable of sufficiently enjoying the effect of cannabinoid can be produced by a powder tea composition with a cannabinoid-containing water-soluble granulated product and a tea leaf powder.

### Example 2

A glass vial container having an inner volume of 50 cc was charged with 5% or 1% of a commercially available water-soluble CBD powder (granulated product granulated mainly with polysaccharide as a binder, repose angle: about 30°, CBD: 2%), and then charged with 95% or 99% of a domestically produced roasted green tea powder, and these were mixed by vibration and rotation of the container at room temperature for about 10 minutes, to obtain 5 g of a powder tea composition. The degree of dispersion was calculated by the same method as described above, and was each more than 99%, or 94%.

Hereinafter, variations of the invention according to the present disclosure are described.

[Note 1]A powder tea composition in a powder form, comprising a cannabinoid-containing water-soluble granulated product and a tea leaf-ground product.

[Note 2] The powder tea composition according to Note 1, wherein a content rate of the water-soluble granulated product is 1% by mass or more based on a total amount of 100% by mass of the powder tea composition.

[Note 3] The powder tea composition according to Note 1 or 2, wherein the tea leaf-ground product comprises a tea leaf-ground product of a plant in the genus Camellia of the family Theaceae.

[Note 4] The powder tea composition according to any one of Notes 1 to 3, wherein a repose angle of the water-soluble granulated product is 45° or less.

[Note 5] The powder tea composition according to any one of Notes 1 to 4, wherein a repose angle of the water-soluble granulated product is 35° or less.

[Note 6] The powder tea composition according to any one of Notes 1 to 5, wherein the powder tea composition is in a core-shell powder form with the water-soluble granulated product as a core layer and the tea leaf-ground product as a shell layer.

[Note 7] The powder tea composition according to any one of Notes 1 to 6, wherein a content rate of the water-soluble granulated product is not less than 1% by mass and not more than 80% by mass based on a total amount of 100% by mass of the powder tea composition.

[Note 8] The powder tea composition according to any one of Notes 1 to 7, wherein a content rate of the water-soluble granulated product is not less than 1% by mass and not more than 70% by mass based on a total amount of 100% by mass of the powder tea composition.

[Note 9] The powder tea composition according to any one of Notes 1 to 8, wherein a content rate of the water-soluble granulated product is not less than 1% by mass and not more than 60% by mass based on a total amount of 100% by mass of the powder tea composition.

[Note 10] The powder tea composition according to any one of Notes 1 to 9, wherein a content rate of the tea leaf-ground product is not less than 20% by mass and not more than 99% by mass based on a total amount of 100% by mass of the powder tea composition.

[Note 11] The powder tea composition according to any one of Notes 1 to 10, wherein a content rate of the tea leaf-ground product is not less than 40% by mass and not more than 95% by mass based on a total amount of 100% by mass of the powder tea composition.

[Note 12] The powder tea composition according to any one of Notes 1 to 11, wherein the water-soluble granulated product is a water-soluble granulated product in which phase separation is not confirmed even after a lapse of 2 hours at room temperature from the time of mixing with water.

[Note 13] The powder tea composition according to any one of Notes 1 to 12, wherein the water-soluble granulated product is a water-soluble granulated product in which phase separation is not confirmed even after a lapse of one day at room temperature from the time of mixing with water.

[Note 14] The powder tea composition according to any one of Notes 1 to 13, wherein a content rate of cannabinoid in the water-soluble granulated product is not less than 0.1% by mass and not more than 60% by mass based on a total amount of 100% by mass of the water-soluble powder.

[Note 15] The powder tea composition according to any one of Notes 1 to 14, wherein the water-soluble granulated product comprises polysaccharide.

[Note 16]A method for producing the powder tea composition according to any one of Notes 1 to 15, the method comprising mixing/stirring a composition including a cannabinoid-containing water-soluble granulated product and a tea leaf-ground product, by a container movement system.

1. A powder tea composition in a powder form, comprising a cannabinoid-containing water-soluble granulated product and a tea leaf-ground product.

2. The powder tea composition according to claim 1, wherein a content rate of the water-soluble granulated product is 1% by mass or more based on a total amount of 100% by mass of the powder tea composition.

3. The powder tea composition according to claim 1, wherein the tea leaf-ground product comprises a tea leaf-ground product of a plant in the genus Camellia of the family Theaceae.

4. The powder tea composition according to claim 2, wherein the tea leaf-ground product comprises a tea leaf-ground product of a plant in the genus Camellia of the family Theaceae.

5. The powder tea composition according to claim 1, wherein a repose angle of the water-soluble granulated product is 45° or less.

6. The powder tea composition according to claim 2, wherein a repose angle of the water-soluble granulated product is 45° or less.

7. The powder tea composition according to claim 3, wherein a repose angle of the water-soluble granulated product is 45° or less.

8. The powder tea composition according to claim 4, wherein a repose angle of the water-soluble granulated product is 45° or less.

9. The powder tea composition according to claim 1, wherein a repose angle of the water-soluble granulated product is 35° or less.

10. The powder tea composition according to claim 1, wherein the powder tea composition is in a core-shell powder form with the water-soluble granulated product as a core layer and the tea leaf-ground product as a shell layer.

11. The powder tea composition according to claim 2, wherein a content rate of the water-soluble granulated product is not less than 1% by mass and not more than 80% by mass based on a total amount of 100% by mass of the powder tea composition.

12. The powder tea composition according to claim 11, wherein the content rate of the water-soluble granulated product is not less than 1% by mass and not more than 70% by mass based on a total amount of 100% by mass of the powder tea composition.

13. The powder tea composition according to claim 11, wherein the content rate of the water-soluble granulated product is not less than 1% by mass and not more than 60% by mass based on a total amount of 100% by mass of the powder tea composition.

14. The powder tea composition according to claim 2, wherein a content rate of the tea leaf-ground product is not less than 20% by mass and not more than 99% by mass based on a total amount of 100% by mass of the powder tea composition.

15. The powder tea composition according to claim 14, wherein the content rate of the tea leaf-ground product is not less than 40% by mass and not more than 95% by mass based on a total amount of 100% by mass of the powder tea composition.

16. The powder tea composition according to claim 1, wherein the water-soluble granulated product is a water-soluble granulated product in which phase separation is not confirmed even after a lapse of 2 hours at room temperature from the time of mixing with water.

17. The powder tea composition according to claim 1, wherein the water-soluble granulated product is a water-soluble granulated product in which phase separation is not confirmed even after a lapse of one day at room temperature from the time of mixing with water.

18. The powder tea composition according to claim 1, wherein a content rate of cannabinoid in the water-soluble granulated product is not less than 0.1% by mass and not more than 60% by mass based on a total amount of 100% by mass of the water-soluble powder.

19. The powder tea composition according to claim 1, wherein the water-soluble granulated product comprises polysaccharide.

20. A method for producing the powder tea composition according to claim 1, the method comprising mixing/stirring a composition including a cannabinoid-containing water-soluble granulated product and a tea leaf-ground product, by a container movement system.
